(19) United States
(12) Patent Application Publication
(10) Pub. No.: US 2022/0379145 A1
(43) Pub. Date: Dec. 1, 2022

(54) RE-USABLE ELASTOMERIC RESPIRATOR AND VENTILATOR WITH FILTER INSERTS AND PORTS

(71) Applicant: West Virginia Military Authority, Charleston, WV (US)

(72) Inventors: Dwight A. Siemiaczko, Charleston, WV (US); Richard Abraham, Chapmanville, WV (US); Gary Hodge, East Bank, WV (US)

(21) Appl. No.: 17/804,605

(22) Filed: May 31, 2022

Related U.S. Application Data
(60) Provisional application No. 63/194,415, filed on May 28, 2021.

(51) Int. Cl. A62B 23/02; A61M 16/14; A61M 16/10; A62B 9/02; B01D 46/00; B01D 46/58; A41D 13/11

(52) U.S. Cl. CPC A62B 23/02; A61M 16/147; A61M 16/107; A62B 9/022; B01D 46/0005; B01D 46/58; A41D 13/1138; B01D 2265/06; B01D 2271/02; B01D 2279/40

(57) ABSTRACT

A reusable, elastomeric respirator and ventilator, with filter inserts, including a mask body having a pliant facial interface on the interior of the mask body, and two apertures centrally positioned on the front face of the mask body. Two interchangeable plugs or ports are provided to be received within and seal the apertures, deliver oxygen into the mask, or deliver liquids to a wearer of the mask. On opposing sides of the mask body are air filter housings which support an air filter, each air filter housing including a base support structure affixed to the mask body and a top securing structure removably secured to the base support structure. One or more straps are affixed to the mask body to secure the mask body about a wearer's head.

### BRIEF SUMMARY

The disclosed technology regards a re-usable elastomeric respirator and ventilator with filter inserts and ports for delivering oxygen and liquids.

### BACKGROUND

Traditional half-face respirators utilize an exhalation valve allowing for a wearer's exhalation to pass through the valve, bypassing the filter, to the environment, thereby passing potentially contagious aerosol from the wearer into the environment. Similarly, ventilators do not filter a wearer's exhalation to protect the surrounding environment from contagious aerosol.

The respirator of the disclosed technology filters air drawn from the environment on inhalation, and filters air exhaled by a wearer into the environment, protecting both the wearer and individuals in the immediate environment from contagious aerosol. The respirator of the disclosed technology further includes ports for the provision of oxygen and/or liquids to the wearer.

### GENERAL DESCRIPTION

The disclosed technology provides a reusable, elastomeric respirator and ventilator, with filter inserts. Generally, the reusable respirator includes a mask body having a pliant facial interface on the interior of the mask body, and two apertures centrally positioned on the front face of the mask body. Two interchangeable plugs or ports are provided to be received within and seal the apertures, deliver oxygen into the mask, or deliver liquids to a wearer of the mask. On opposing sides of the mask body are air filter housings which support an air filter, each air filter housing including a base support structure affixed to the mask body and a top securing structure removably secured to the base support structure. One or more straps are affixed to the mask body to secure the mask body about a wearer's head.

The disclosed technology further provides a reusable elastomeric respirator including a mask body having a pliant facial interface on the interior of the body, and two apertures centrally positioned on the front face of the mask body. Two plugs or ports are included to seal the apertures, deliver oxygen into the mask, or deliver liquids to a wearer of the mask. The plugs or ports include a hollow stem with an interior collar, a ring and a face, all secured circumferentially about the exterior of the stem. Air filter housings are secured to opposing sides of the mask body and support an air filter, each air filter housing including a base support structure affixed to the mask body and a top securing structure removably secured to the base support structure. The base support structures each includes an underframe and an upperframe with aligned apertures, wherein edges of an aperture of the mask body are secured between perimeter, adjoining surfaces of the underframe and the upperframe. The upperframe further has a solid surface about its perimeter, with a plurality of internal support structures extending up from the solid surface to support the air filter a distance above the solid surface, without significantly impeding airflow through the air filter. One or more straps are secured to eyelets extending from an end of each of the top securing structures to secure the mask body about a wearer's head.

These and other advantages of the present disclosure are more fully demonstrated in the illustrations and detailed descriptions. Various embodiments may take on other modifications and alterations without departing from the spirit or scope of the disclosure as described in the illustrations above. Accordingly, this disclosure is not to be limited to the above described illustrations, but rather by the limitations set forth in the following claims and any equivalents thereof provided.

### DESCRIPTION OF THE FIGURES

Features of the present disclosure will become more fully apparent from the following description and appended claims, taken in conjunction with the accompanying drawings. It will be understood these drawings depict only certain embodiments in accordance with the disclosure and, therefore, are not to be considered limiting of its scope; the disclosure will be described with additional specificity and detail through use of the accompanying drawings. An apparatus, system or method according to some of the described embodiments can have several aspects, no single one of which necessarily is solely responsible for the desirable attributes of the apparatus, system or method. After considering this discussion, and particularly after reading the section entitled “Detailed Description” one will understand how illustrated features serve to explain certain principles of the present disclosure.

FIG. 1 shows a front view of an embodiment of the fully assembled re-usable elastomeric respirator and ventilator of the disclosed technology;

FIG. 2 shows a front view of the embodiment of FIG. 1, with the top securing structure and straps removed;

FIG. 3 shows an interior view of the embodiment of FIG. 1, with the head and neck straps removed;

FIG. 4A shows a side view, and FIG. 4B shows a peripheral view, of the embodiment of FIG. 1, with one of the top securing structures and the straps removed;

FIG. 4C shows a front view of the embodiment of FIG. 1, with the straps removed;

FIG. 5 is a side view of a closed plug of the disclosed technology;

FIG. 6 is a side view of an oxygen port of the disclosed technology;

FIG. 7 is a side view of a liquids port of the disclosed technology;

FIG. 8 is an exterior view of the base support structure of a filter housing of the embodiment of FIG. 1, as affixed to a mask body;

FIG. 9 is an interior view of the base support structure of a filter housing of the embodiment of FIG. 1, as affixed to a mask body;

FIG. 10 is an interior view of a top securing structure of a filter housing of the embodiment of FIG. 1;

FIG. 11 is an exterior view of a top securing structure of a filter housing of the embodiment of FIG. 1;

FIG. 12 is a peripheral view of a head strap of the embodiment of FIG. 1;

FIG. 13 is a peripheral view of a portion of a neck strap of embodiment of FIG. 1; and

FIG. 14 depicts fastener means useful for securing the head strap or the neck strap to the top securing structure in the embodiment of FIG. 1.

### DETAILED DESCRIPTION

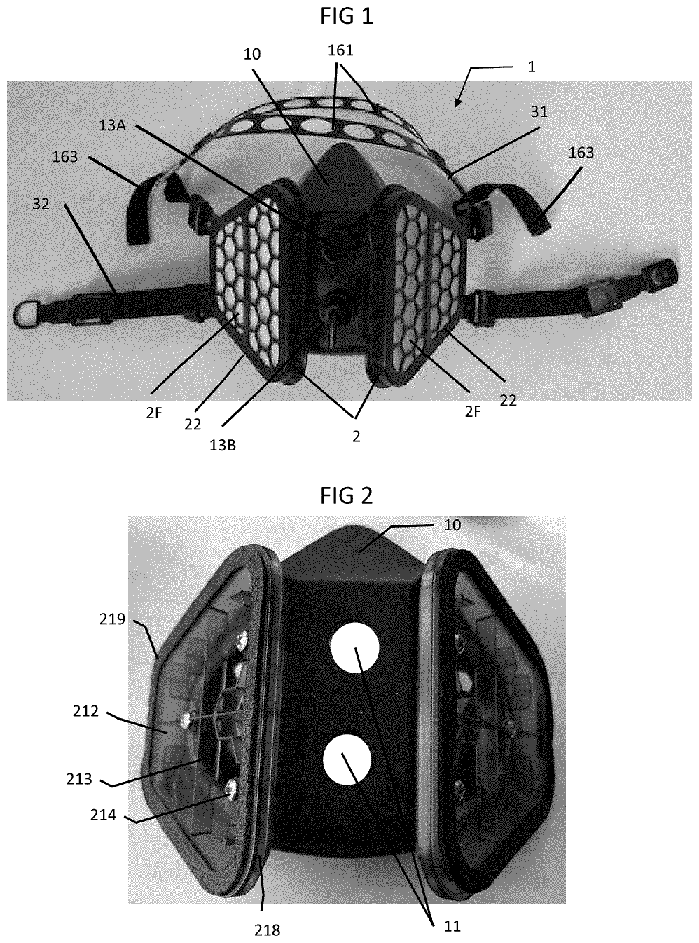

As shown in the embodiments of the drawings hereinabove described and provided with this disclosure, the disclosed technology provides a reusable elastomeric respirator 1 also usable as a ventilator, and further capable of delivering liquids to a wearer. Generally, as shown in FIGS. 1-4C, the disclosed technology includes a mask body 10, having a pliant facial interface 12 on the interior of the body, one or more apertures 11 centrally positioned on the front face of the mask body, and a pair of air filter housings 2 secured to opposing sides of the mask body. The mask body may be made from thermoplastic elastomers (TPE), silicone, or like materials

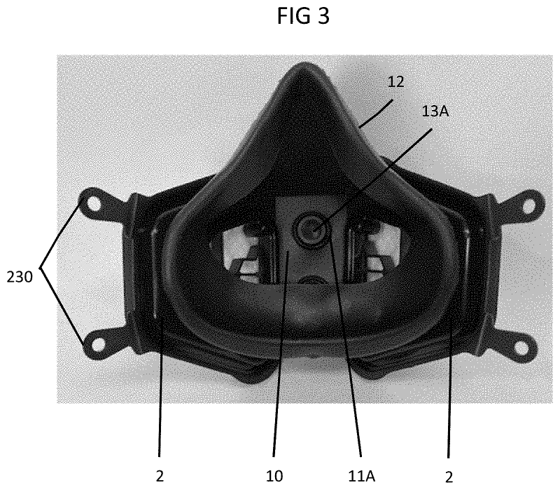

The pliant facial interface 12 on the interior of the mask body forms a leak free seal between the mask body and the wearer's face, over the nose and mouth. The pliant facial interface may be secured to the mask body, or may be integral with the mask body as shown in FIG. 3. As with the mask body, the interface may be made from TPE, silicone, or like materials. Applicant uses a TPE manufactured by Avient Corporation under the product designation LC AC7-022 for both the mask body and the facial interface.

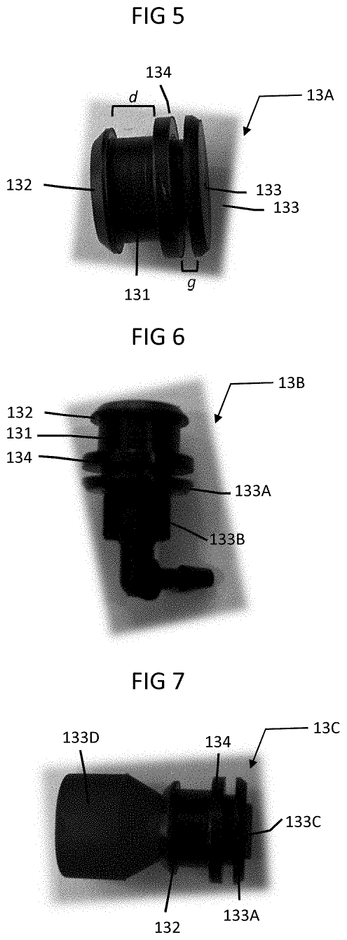

As shown in particular in FIG. 2, extending through the central portion of the face of the mask body are one or more apertures 11 which may receive a closed plug 13A (see FIG. 5), an oxygen port 13B (see FIG. 6) for delivering oxygen to a wearer, or a liquids port 13C (see FIG. 7) for delivering liquids, such as water and liquid food, to the wearer. As shown in FIG. 3, the interior surface of the mask body may have collars 11A to lengthen the apertures 11 and support the stem of the plug or port.

Referring to FIG. 5, the closed plug 13A includes a hollow stem 131, an interior collar 132, and a solid face 133. A ring 134 is also positioned along and affixed to the outer surface of the hollow stem, leaving a gap g along the hollow stem and between the ring and the solid face, suitable for receiving a supporting edge of a face shield. The hollow stem in the gap may have a thickness less than the thickness of the remainder of the hollow stem, to provide greater depth in the gap for receiving and supporting the face shield; alternatively, the radii of the ring and plug could be increased to provide such depth. When used, the face shield has an aperture or supporting edge sized and positioned to receive the plug or port and support the face shield over the wearer's face.

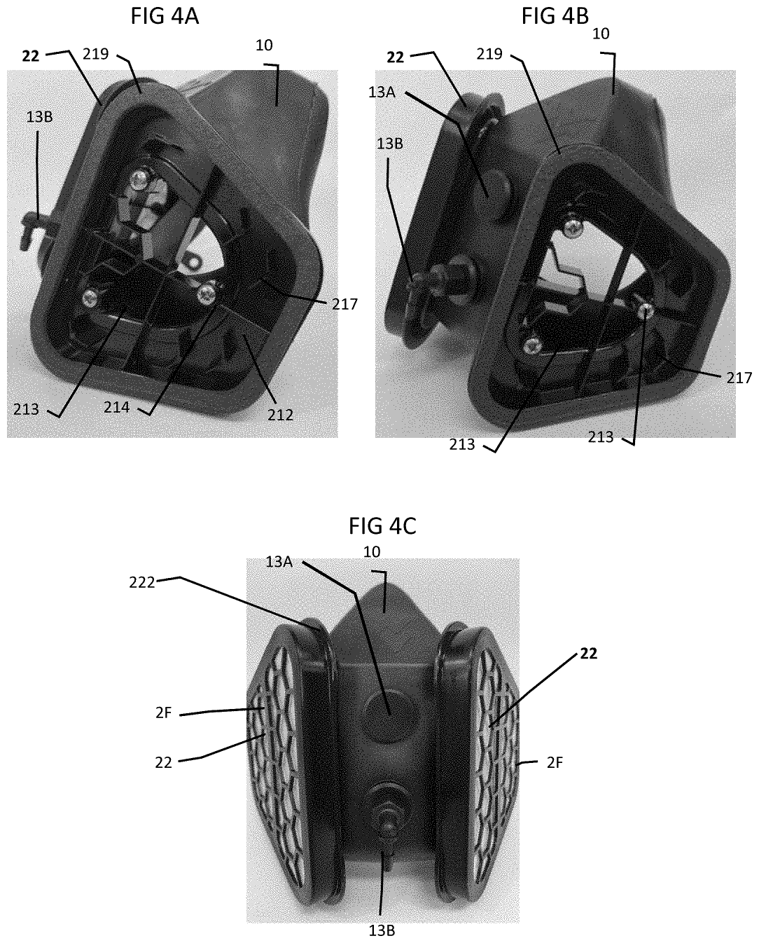

The depth d of the hollow stem 131 along its length between the interior collar 132 and the ring 134, is about the same as the length of the apertures 11 as lengthened by the collars 11A, so that the surface of the mask body at the apertures is in contact with and supports the stem when positioned therewithin. The interior collar 132 has a radius larger than the aperture so that it retains the plug within the aperture, but not so large that it cannot be easily positioned within the aperture and removed therefrom. The ring 134 also has a radius greater than the aperture to seal the apertures closed. When secured within the aperture, the interior collar 132 of the plug rests on the upper surface of the collar 11A (see, e.g., FIG. 3), and the solid face or alternatively the ring, if present, rests upon the exterior surface of the mask body (see, e.g., FIG. 4), thereby sealing the aperture closed. To assist with securing and removing the plug in the aperture, the interior collar 132 may have a sloped surface, receding in radius from its proximal edge to its distal edge, as shown in FIG. 5.

Likewise, as shown in FIG. 6 the oxygen port 13B has the same configuration as the closed plug 13A as hereinabove described, except that the solid face 133A has an aperture therethrough and an air line fitting (shown with a barb coupling) 133B extending therefrom, suitable for receiving and removably securing an air line from an oxygen supply.

As shown in FIG. 7, the liquids port 13C is similarly constructed to the design of the closed plug, except the solid face 133A has an aperture therethrough, extending the hollow stem to the face of the port, wherein the hollow stem 131 receives and supports a bite valve 133C therethrough. The bite valve has a soft (silicone) cap 133D at one end, and a liquid line can extend from, be securely received in or coupled with the opposing end of the bite valve. Other ports for these or other purposes could be similarly constructed to be received within the apertures 11 on the front face of the mask body.

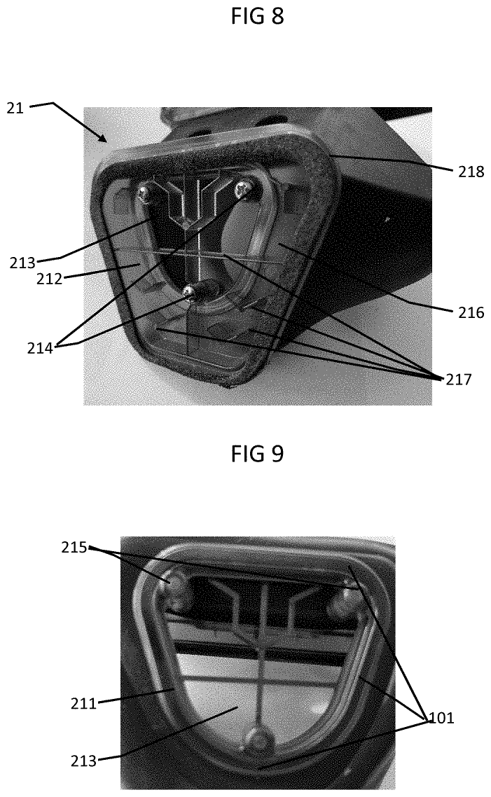

Referring back to FIGS. 1-4C, and further to FIGS. 8-11, affixed on opposing sides of the mask body are air filter housings 2 for receiving and retaining an air filter 2F, such as an N95 filter. Each filter housing includes a base support structure 21 and a removable or hinged top securing structure 22. The base support structure includes an underframe 211 and an upperframe 212, having aligned apertures 213 to allow airflow through the filter housings. The underframe and upperframe can be secured together by means of fasteners, such as screws 214 received in threaded ports 215 integrally formed on or otherwise secured to the underframe, wherein the mask body has apertures to accommodate the base support structure, the edges 101 of the mask body forming such apertures being secured between the adjoining surfaces of the underframe and the upperframe of the base support structure, thereby creating a tight seal, and securing the base support structure to the mask body.

The upperframe has a solid surface 216 about its perimeter, the solid surface supporting a plurality of internal support structures 217 to support the air filter about ¼″ to ½″ above the solid surface, thereby facilitating the flow of air through the entire filter. Further, the upperframe has a collar 218 about its a top perimeter. The collar may have a depth of between ¼ and ¾ of the depth of the upperframe. The upperframe of the base support structure may include a foam gasket 219 affixed to the top surface of the collar 218, thereby facilitating compression of the edges of the filter to the filter housing, ensuring a secure fit. See FIGS. 2, 4A, 4B and 9.

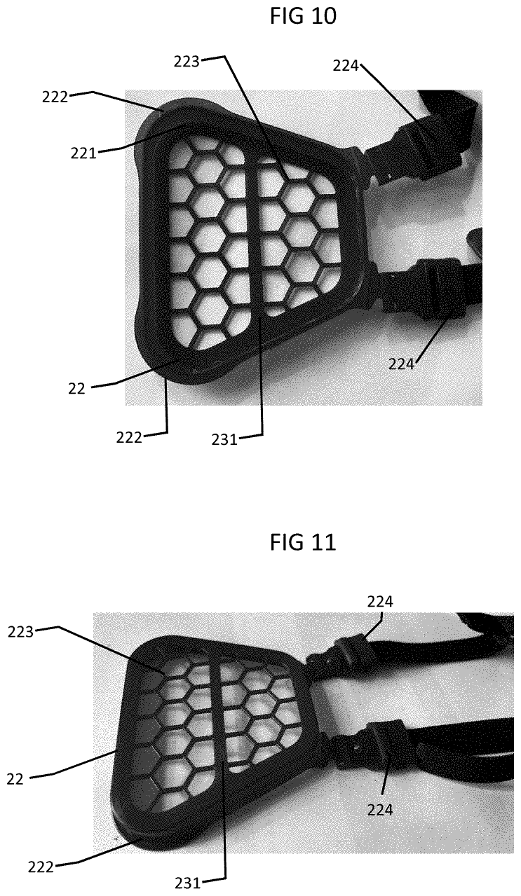

As shown in FIGS. 10 and 11, the top securing structure 22 includes a frame which secures about the collar 218 of the upperframe, by means of a ridge 221 about the frame's lower interior perimeter, for example. One or more flanges 222 may extend from the exterior of the frame to facilitate removal of the top securing structure from the filter housing for replacement of the filter.

Alternatively, a hinge may be incorporated into one side of both the base support structure and the top securing structure, with a clip or other securing means on the opposing side, to removably secure the top securing structure to the base support structure.

As shown in FIGS. 1, 10 and 11, the top securing structure 22 is intended to allow the wearer to breath in and out through the filter; therefore, a webbing 223 and minimal support structure 231 is provided within and supported by the frame, sufficient to retain the filter without inhibiting the wearer's ability to breathe.

By these configurations of the filter housing and the various components thereof, the air filter may be easily inserted, removed and replaced.

The base support structure underframe and upperframe of the filter housing may be made from rigid materials such as polycarbonate, which may be transparent to allow assemblers to see the placement of the air filter. The applicant has used Lotte Infino SC 122 clear polycarbonate prime 20MF purchased from Texco Resinm Distribution Co. Alternatively, they may be made from more flexible materials which are also suitable for the top securing structure, such as, for example, a Generic Prime Homopolymer Polypropylene 12MF resin purchased from Texco Resin Distribution Co.

As shown in FIGS. 1 and 3, the air filter housings 2 provides means for supporting an adjustable head strap 31, and can further provide means for supporting an adjustable neck strap 32, to secure the mask body to a wearer's head, thereby insuring a sealed fit between the pliant facial interface and the wearer's face.

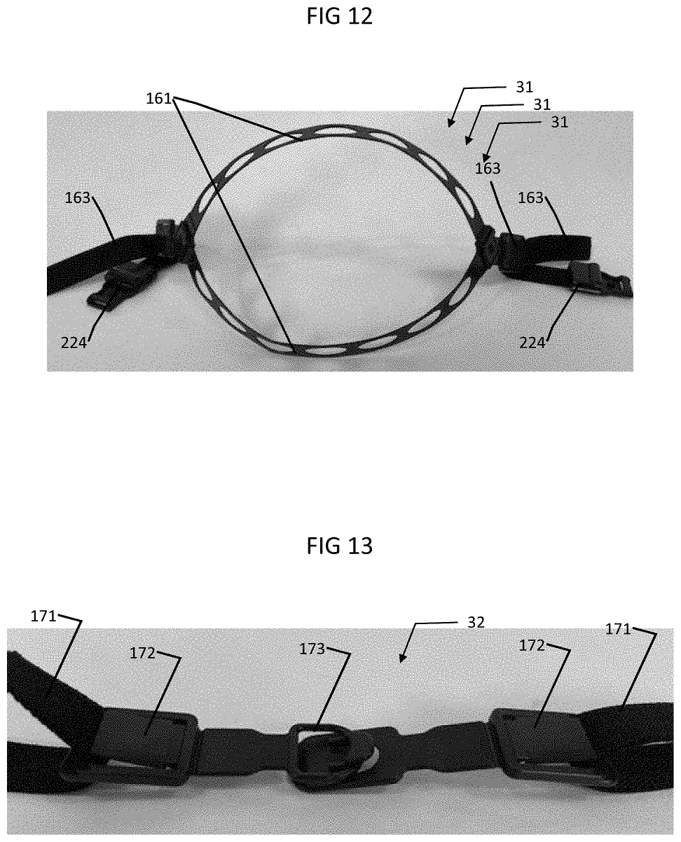

As shown in FIGS. 1 and 12, the head strap may include one or more flexible straps 161, such as flexible webbing as shown the figures, and material straps 163. In the embodiment shown, two flexible straps are secured at their ends at an angle (e.g., a 90° angle), to provide a secure and comfortable fit about a wearer's head. This webbing may be made from polypropylene, such as that described above for the top securing structure, or other flexible material. Extending from the ends of the straps 161 is a strap adjuster 162, to receive a material strap 163, allowing the length of the head strap to be adjusted while securing it in place after adjustment.

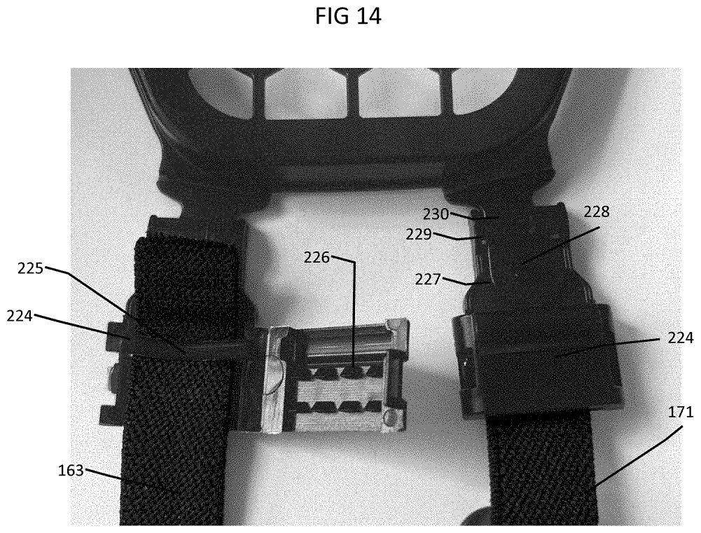

To secure the head strap to the filter housings, fastener means are provided to receive a respective distal end of each of the material straps 163. In the embodiment shown in FIG. 14, the fastener means comprises a clip 224, having a post 225 about which the material straps may be threaded and folded over, and teeth 226 to secure and hold the material within the clip when closed. Extending from an end of each clip is a tray 227, having a short, slanted post 228 centrally located thereon and a retaining bar 229, to receive an eyelet 230 of the top securing structure, wherein when secured the eyelet is received in the tray, under the retaining bar, and the aperture of the eyelet sized to receive the short, slanted post. By this configuration and the size of the tray relative to the size of the eyelet, the head strap is rotatably secured to the top securing structure, and thereby the respirator of the disclosed technology when assembled. As with other elements of the disclosed technology, polypropylene material is suitable for purposes of these fastening components. Other means for affixing the head strap to the filter housings and the mask body may also be used.

A neck strap as shown in FIGS. 1 and 13 may likewise be provided, including for example a pair of material straps 171, adjustable by means of one or more strap adjustors 172, and removably secured by means of a buckle or clip 173. The opposing sides of the neck strap are removably or fixedly secured to the top securing structure of the filter housings, wherein fastener means as hereinabove described and shown in FIG. 14 are provided at each end of the neck strap, to rotatably secure the neck strap to the eyelets of the top securing structure.

Replaceable air filters 2F, such as N95 filters, are sized to fit within the filter housing, and can be placed and replaced therein when the top securing structure is removed from the base securing structure.

As hereinabove described, when assembled and secured to a wearer, the mask of the disclosed technology filters the wearer's inhalation and exhalation without the use of an exhalation valve, providing protection to both the wearer and individuals in the immediate vicinity of the wearer.

While this invention has been described in connection with what are presently considered to be practical exemplary embodiments, it will be appreciated by those skilled in the art that various modifications and changes may be made without departing from the scope of the present disclosure. It will also be appreciated by those of skill in the art that parts included in one embodiment are interchangeable with other embodiments; one or more parts from a depicted embodiment can be included with other depicted embodiments in any combination. For example, any of the various components described herein and/or depicted in the Figures may be combined, interchanged or excluded from other embodiments. With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. Thus, while the present disclosure has described certain exemplary embodiments, it is to be understood that the invention is not limited to the disclosed embodiments, but, on the contrary, is intended to cover various modifications and equivalent arrangements included within the spirit and scope of the appended claims.

1. A reusable elastomeric respirator comprising:
a. a mask body having a pliant facial interface on the interior of the mask body, and two apertures centrally positioned on the front face of the mask body;
b. two plugs or ports to seal the apertures, deliver oxygen into the mask, or deliver liquids to a wearer of the mask, the plugs or ports being positioned within the apertures of the mask body;
c. two air filter housings secured to opposing sides of the mask body and supporting an air filter, each air filter housing comprising a base support structure affixed to the mask body and a top securing structure having a frame, which top securing structure is removably secured to the base support structure; and
d. one or more straps to secure the mask body about a wearer's head.

2. The reusable elastomeric respirator of claim 1, wherein the base support structures of the air filter housings each includes an underframe and an upperframe with aligned apertures to allow airflow through the filter housing.

3. The reusable elastomeric respirator of claim 2, wherein edges of an aperture of the mask body is received and secured between adjoining surfaces of the underframe and the upperframe of the base support structure, thereby creating a tight seal and securing the base support structure to the mask body.

4. The reusable elastomeric respirator of claim 2, wherein the upperframe has a solid surface about its perimeter, supporting a plurality of internal support structures to support the air filter above the solid surface, thereby facilitating the flow of air through the entire filter.

5. The reusable elastomeric respirator of claim 2, wherein the upperframe has a collar about its top perimeter and the top securing structure secures about the collar of the upper frame by means of a ridge about an interior surface of its frame, and wherein one or more flanges are affixed to the exterior of the top securing structure frame to facilitate removal of the top securing structure from the base support structure.

6. The reusable elastomeric respirator of claim 5, Wherein the upperframe comprises a foam gasket affixed to a top surface of the upperframe collar.

7. The reusable elastomeric respirator of claim 1, wherein the top securing structure comprises webbing secured within the frame to retain the filter in the air filter housing.

8. The reusable elastomeric respirator of claim 1, wherein one of the one or more straps is an adjustable head strap.

9. The reusable elastomeric respirator of claim 8, wherein the adjustable head strap comprises two flexible webbing straps with the ends of each strap being secured to the ends of the other strap, at an angle, and further comprises two material straps removably affixed to each of the secured ends of the webbing straps by means of a strap adjuster.

10. The reusable elastomeric respirator of claim 8, wherein a second of the one or more straps is a neck strap comprised of a pair of material straps adjustable by means of one or more strap adjustors, and removably secured to each other by means of a buckle or clip.

11. The reusable elastomeric respirator of claim 1, wherein the one or more straps are secured to eyelets extending from an end of each of the top securing structures.

12. The reusable elastomeric respirator of claim 11, wherein the one or more straps are secured to the eyelets of the top securing structures by fastener means comprising a clip, having a post about which the material straps may be threaded and folded over, and teeth to secure and hold the material within the clip when closed, wherein extending from an end of each clip is a tray having a short, slanted post centrally located thereon and a retaining bar, to receive and secure the eyelet of the top securing structure, wherein when secured the eyelet is received in the tray, under the retaining bar, and an aperture of the eyelet receives the short, slanted post.

13. The reusable elastomeric respirator of claim 1, wherein the mask body is made from thermoplastic elastomers (TPE).

14. The reusable elastomeric respirator of claim 1, wherein collars are positioned on the interior of the mask body, aligned with the apertures, to lengthen the apertures.

15. The reusable elastomeric respirator of claim 1, wherein the plug or port comprises a hollow stem with an interior collar, a ring and a face secured about the exterior of the stem, the space between the ring and the face forming a gap to support a securing edge of a face shield.

16. The reusable elastomeric respirator of claim 15, wherein the face of the plug or port has an aperture and either an air line fitting extending from its surface and aligned with the aperture, or a bite valve with a silicone cap.

17. The reusable elastomeric respirator of claim 15, wherein the length of the hollow stem between the interior collar and the ring is about the same as the length of the aperture of the mask body, as lengthened by the collars.

18. The reusable elastomeric respirator of claim 15, wherein each of the interior collars and the rings of the plug or port has a radius larger than the radius of the apertures of the mask body, to removably secure the same within and seal the apertures.

19. The reusable elastomeric respirator of claim 15, wherein the interior collar has a sloped surface, receding in diameter from its proximal edge to its distal edge.

20. A reusable elastomeric respirator comprising:
a. a mask body having a pliant facial interface on the interior of the mask body, and two apertures centrally positioned on the front face of the mask body;
b. two plugs or ports to seal the apertures, deliver oxygen into the mask, or deliver liquids to a wearer of the mask, the plugs or ports being positioned within the apertures of the mask body, wherein each plug or port comprises a hollow stem with an interior collar, a ring and a face secured about the exterior of the stem;
c. two air filter housings secured to opposing sides of the mask body and supporting an air filter, each air filter housing comprising a base support structure affixed to the mask body and a top securing structure removably secured to the base support structure, wherein the base support structures each includes an underframe and an upperframe with aligned apertures and wherein edges of an aperture of the mask body is received and secured between adjoining surfaces of the underframe and the upperframe, and wherein the upperframe has a solid surface about its perimeter, having a plurality of internal support structures to support the air filter a distance above the solid surface; and
d. one or more straps secured to eyelets extending from one end of each of the top securing structures to secure the mask body about a wearer's head.
